(19) United States
(12) Patent Application Publication
(10) Pub. No.: US 2017/0231356 A1
(43) Pub. Date: Aug. 17, 2017

(54) COSMETIC TREATMENT OF KERATIN FIBRES

(71) Applicant: L'OREAL, Paris (FR)

(72) Inventors: Henri SAMAIN, Bievres (FR); Franck GIRON, Lagny sur Marne (FR); Philippe ILEKTI, Maisons-Alfort (FR)

(73) Assignee: L'OREAL, Paris (FR)

(21) Appl. No.: 15/314,978

(22) PCT Filed: May 29, 2015
(86) PCT No.: PCT/IB2015/054052

§ 371 Date: Nov. 30, 2016

(30) Foreign Application Priority Data
May 30, 2014 (FR) 1454907

(51) Int. Cl. A45D 19/02; A61K 8/92; A45D 2/48; A61K 8/85; A41G 5/02; A45D 34/04; A61Q 1/10; A61K 8/81

(52) U.S. Cl. CPC A45D 19/02; A61Q 1/10; A61K 8/922; A61K 8/8152; A61K 8/85; A41G 5/02; A45D 34/04; A45D 2/48; A61K 2800/87

(57) ABSTRACT

The present invention is directed towards a cosmetic assembly, of use for the treatment of keratin fibres, comprising a fluid cosmetic composition containing at least particles in the dispersed state in a liquid medium, said composition being solidifiable via the aggregation of said particles; and a device (1) comprising a mould (2), the mould (2) comprising at least one cavity (5), into which or each of which at least one of said fibres can be at least partially introduced, for the purposes of moulding said composition onto at least one part of said fibre(s) present in the cavity or cavities (5). The present invention is also directed towards a process for cosmetic treatment of one or more keratin fibres.

The present invention aims to provide, for the field of keratin fibre care and/or makeup, a novel treatment which is quite particularly advantageous from the viewpoint of the new technical performance levels that it confers.

The term “keratin fibres” is intended to mean preferably human keratin fibres, in particular the eyelashes, the eyebrows, body hair and the hair, and preferentially the eyelashes or the eyebrows.

Generally, keratin fibres, and more particularly eyelashes, are commonly made up in order to confer thereon greater visibility and/or an original appearance.

In order to achieve this objective, the makeup technique most used is the application of mascara. The latter is generally effective, but it remains, however, to be improved in terms of gain in visibility.

To date, this gain has essentially been obtained by increasing the amount of material deposited on the eyelashes via an increased repetition of the number of brushstrokes dedicated to the application of the composition to the surface of the eyelashes. However, this solution is not satisfactory.

Indeed, at each new deposit, material is also removed. In addition, the creation of unattractive overloads and/or agglomerates is generally observed.

Finally, this makeup method does not make it possible to obtain certain aesthetic effects which are highly appreciated by users, for instance the obtaining of a coating on the eyelashes which is very smooth, or even has an advantageous gloss provided by this smoothing effect.

The documents FR 2 936 420 A1, FR 2 923 381 A1, US 2007/286831 A1 and WO 2006/043544 A1 disclose application of mascara with mascara brushes.

Another makeup technique known to increase eyelash visibility is based on the use of additional fibres, for example false eyelashes, which are in particular stuck to the natural eyelashes.

A first variant of this technique consists in sticking false eyelash fringes to the eyelid. Such false eyelash fringes, and also the processes for producing them, are described in patents U.S. Pat. No. 2,421,432 and U.S. Pat. No. 3,559,657. Nevertheless, putting them on remains difficult and the result remains not very natural. In addition, persistence is weak, and generally limited to a day.

A second variant of this technique consists in sticking false eyelashes one by one to the eyelashes. Although longer lasting, this technique is expensive, takes a long time to perform, and can only be carried out by an individual who works in the profession.

There remains therefore a need for a makeup method which makes it possible to obtain effects of large volume and/or of great length, and which does not have the abovementioned drawbacks. In particular, there remains a need for a method for making up keratin fibres which makes it possible to easily and rapidly create new effects on the keratin fibres, including the smooth and glossy coating effect, of sheath type.

The invention aims specifically to meet these needs.

Thus, a subject of the present invention is a cosmetic assembly, of use for the treatment of keratin fibres, in particular the eyelashes and the eyebrows, comprising:
a fluid cosmetic composition containing at least particles in the dispersed state in a liquid medium, said composition being solidifiable via the aggregation of said particles; and
a device (1) comprising a mould (2), the mould (2) comprising at least one cavity (5), and preferably several cavities (5), into which or each of which at least one of said fibres can be at least partially introduced for the purposes of moulding said composition onto at least one part of said fibre(s) present in the cavity or cavities (5).

Thus, a composition according to the invention sets solid by aggregation of the particles dispersed in the liquid medium under the action of an increase in the temperature and/or by evaporation of said liquid medium.

Against all expectations, the invention makes it possible to accurately and reproducibly control the shape of the deposit of composition on the fibres treated, regardless of the dexterity of the individual. Furthermore, this makeup method makes it possible to obtain makeup results thus far unobtainable using conventional techniques. In particular, it makes it possible to significantly extend the length of the keratin fibres and to increase the thickness of the makeup deposit, while providing a smooth and homogeneous, or even glossy, effect. Moreover, it also makes it possible to integrate, into the sheath of makeup produced at the surface of the eyelashes, additional fibres like those considered for false eyelashes.

According to one preferred embodiment, the particles in the dispersed state comprise at least one latex, i.e. at least one film-forming polymer in the form of particles in dispersion, preferably aqueous dispersion.

According to one preferred embodiment, the particles in the dispersed state comprise at least one latex and at least one wax, preferably at least one polar wax, in particular at least one carnauba wax, for example introduced in the form of an aqueous dispersion.

To do this, a composition may comprise a total solids content of particles in the dispersed state of greater than or equal to 5% by weight, preferably greater than or equal to 10% by weight, and better still greater than or equal to 12% by weight, relative to the total weight of the composition, more preferentially a total solids content of particles in the dispersed state ranging from 10% to 55% by weight, in particular from 12% to 50% by weight, relative to the total weight of the composition.

According to another of its aspects, a subject of the invention is also a process for cosmetic treatment of one or more keratin fibres, in particular of one or more eyelashes or eyebrows, comprising at least the step consisting in moulding a fluid cosmetic composition, containing at least particles in the dispersed state in a liquid medium, said composition being solidifiable via the aggregation of said particles, onto at least one part of said fibres, by means of one or more cavities (5), of a mould (2), into which said fibres are at least partially introduced.

According to yet another of its aspects, the present invention relates to the use of a fluid cosmetic composition containing at least particles in the dispersed state in a liquid medium, said composition being solidifiable via the aggregation of said particles, for application thereof as a moulding material in at least one cavity of a mould, for the purposes of depositing on keratin fibres, in particular eyelashes and eyebrows, via the application of said mould to said fibres.

Cosmetic Composition

As previously mentioned, a cosmetic composition according to the invention is fluid at ambient temperature and contains at least particles in the dispersed state in a liquid medium.

For the purposes of the invention, the description “fluid” is intended to characterize the fact that a composition according to the invention is not solid. In other words, it manifests a fluidity sufficient to have flow properties.

Thus, a composition according to the invention may advantageously have a viscosity of less than 100 Pa·s, preferably between 0.01 Pa·s and 50 Pa·s, preferably between 0.1 Pa·s and 50 Pa·s, and more preferably between 5 Pa·s to 50 Pa·s, at ambient temperature and pressure, the viscosity being in particular measured using a Rheomat RM100®.

In the context of the present invention, it is the reorganization of the particles dispersed in the liquid medium which produces the setting of the particles.

To do this, a composition may comprise from 10% to 55% by weight and preferably from 12% to 50% by weight of particles in the dispersed state, relative to the total weight of the composition.

These particles in the solidstate are present, in the composition according to the invention, in a state dispersed in the associated liquid medium. Thus, a composition according to the invention may be in the form of a colloid or else of a suspension.

As previously mentioned, the cosmetic composition is solidifiable via the aggregation of the particles in the dispersed state.

The particles aggregate under the action of an increase in the temperature and/or by evaporation of the liquid medium. Therefore, the composition goes from a fluid state to a thicker state, and better to a solid state.

For the purposes of the invention, the description “aggregation” is intended to characterize the fact that a sufficiently cohesive material or deposit is obtained, and that such material or deposit may be isolated.

A macroscopically continuous deposit, and preferably cohesive, is thus obtained. More preferably, a reportable deposit which can be individually manipulated is obtained. For example, such deposit may be obtained when the deposit is made by pouring onto a non-stick surface such as a Teflon or silicone surface.

Therefore, the present invention is different from a composition which solidifies by aggregation but which does not become cohesive. For example, a composition according to the present invention is different from a composition consisting in water and pigments which by drying will aggregates. Indeed, the obtained deposit will be powdery but not cohesive.

Thus, according to a preferred embodiment, the fluid cosmetic composition of the cosmetic assembly according to the present invention provides, after its solidification via the aggregation of the particles, a cohesive material or deposit preferably insensitive to water. In particular, such cohesive material or deposit exhibits a water uptake of less than or equal to 10%, preferably less than or equal to 5%, and more preferably less than or equal to 3%.

For the purposes of the invention, the description “water uptake” is understood to mean the percentage of water absorbed by the material or the deposit after 60 minutes of immersion in water at 25° C. (ambient temperature).

The water uptake is measured for a layer of composition 300 μm thick (before drying), laid down with the aid of a 300μ applicator on a sheet of glass equipped with a layer of Teflon-coated tape and then dried at 30° C. for 24 hours on a thermostated plate. Three pieces measuring approximately 1 cm² are cut from the dry film and then weighed (mass measurement M1), and then are immersed in water for 60 minutes; following immersion, the piece of film is wiped to removed the excess surface water and then weighed again (mass measurement M2). The difference M2−M1 corresponds to the amount of water absorbed by the film. The water uptake is equal to [(M2−M1)/M1]×100 and is expressed as a percentage by weight of water relative to the weight of the material or the deposit.

Thus, according to one embodiment variant, the aggregation of the particles dispersed in the liquid medium can be induced by at least partial, or even total, evaporation of said liquid medium.

Preferably, according to this embodiment variant, the evaporation of said liquid medium is carried out at a temperature of between 45° C. and 90° C., preferably between 50° C. and 80° C.

The evaporation of the liquid medium can also be carried out at ambient temperature, by being in particular stimulated by a forced ventilation, using for example a hairdryer.

Thus, a composition according to the invention lends itself to thickening via the partial or total evaporation of the liquid ingredients, most commonly represented predominantly by water.

As mentioned above, a composition according to the invention, subject to this phenomenon of evaporation of the liquid ingredients forming its liquid medium, has the advantage of solidifying to form, in the end, a totally cohesive solid material.

This phenomenon is to be distinguished from a phase transition in the true sense, during the passing from one state of the material to another, such as crystallization.

As previously seen, the cosmetic composition comprises particles in the dispersed state and a liquid medium.

Said liquid medium may be, for example, chosen from water, ethanol or mixtures thereof.

The particles in the dispersed state may be chosen from a wax, a film-forming polymer, and mixtures thereof, and in particular may be chosen from a mixture of a film forming polymer and a wax.

In particular, it may be a question of wax emulsions, a microdispersion of wax in water, a dispersion of wax in an anhydrous solvent or else a film-forming polymer dispersed in water.

Preferably, the cosmetic composition comprises film-forming polymer(s) under the form of particles in the dispersed state and a liquid medium.

Wax

The term “wax” is intended to mean in general a lipophilic compound that is solid at ambient temperature (25° C.), with a reversible solid/liquid change in state, having a melting point of greater than or equal to 30° C., which may be up to 200° C. and in particular up to 120° C.

For the purposes of the invention, the melting point corresponds to the temperature of the most endothermic peak observed in thermal analysis (DSC) as described in the standard ISO 11357-3; 1999. The melting point of the wax may be measured using a differential scanning calorimeter (DSC), for example the calorimeter sold under the name MDSC 2920 by the company TA Instruments.

The measuring protocol is as follows:

A 5 mg sample of wax placed in a crucible is subjected to a first temperature rise ranging from −20° C. to 100° C., at a heating rate of 10° C./minute, it is then cooled from 100° C. to −20° C. at a cooling rate of 10° C./minute and is finally subjected to a second temperature rise ranging from −20° C. to 100° C. at a heating rate of 5° C./minute. During the second temperature rise, the variation in the difference in power absorbed by the empty crucible and by the crucible containing the sample of wax is measured as a function of the temperature. The melting point of the compound is the temperature value corresponding to the top of the peak of the curve representing the variation in the difference in power absorbed as a function of the temperature.

The waxes that may be used in the compositions according to the invention are chosen from waxes that are solid at ambient temperature of animal, vegetable, mineral or synthetic origin, and mixtures thereof.

The waxes, for the purposes of the invention, may be those used generally in the cosmetic or dermatological fields. They may in particular be polar or apolar, and hydrocarbon-based, silicone and/or fluoro waxes, optionally comprising ester, acid or hydroxyl functions. They may also be of natural or synthetic origin.

a) Apolar Waxes

For the purposes of the present invention, the term “apolar wax” is intended to mean a wax of which the solubility parameter at 25° C. as defined below, δₐ, is equal to 0 (J/cm³)^{1/2}.

The definition and calculation of the solubility parameters in the Hansen three-dimensional solubility space are described in the article by C. M. Hansen: The three-dimensional solubility parameters, J. Paint Technol. 39, 105 (1967).

According to this Hansen space:
δ_{D} characterizes the London dispersion forces derived from the formation of dipoles induced during molecular impacts;
δₚ characterizes the Debye interaction forces between permanent dipoles and also the Keesom interaction forces between induced dipoles and permanent dipoles;
δₕ characterizes the specific interaction forces (such as hydrogen bonding, acid/base, donor/acceptor, etc.); and
δₐ is determined by the equation:

δₐ=(δₚ²+δₕ²)^{1/2}.

The parameters δₚ, δ_{b}, δ_{D} and δₐ are expressed in (J/cm³)^{1/2}.

Apolar waxes are in particular hydrocarbon-based waxes constituted solely of carbon and hydrogen atoms, and free of heteroatoms such as N, O, Si and P.

The apolar waxes are chosen from microcrystalline waxes, paraffin waxes, ozokerite and polyethylene waxes, and mixtures thereof.

An ozokerite that may be mentioned is Ozokerite Wax SP 1020 P.

As microcrystalline waxes that may be used, mention may be made of Multiwax W 445® sold by the company Sonneborn, and Microwax HW® and Base Wax 30540® sold by the company Paramelt, and Cerewax® No. 3 sold by the company Baerlocher.

As microwaxes that may be used in the compositions according to the invention as apolar wax, mention may be made in particular of polyethylene microwaxes such as those sold under the names Micropoly 200®, 220®, 220L® and 250S® by the company Micro Powders.

Polyethylene waxes that may be mentioned include Performalene 500-L Polyethylene and Performalene 400 Polyethylene sold by New Phase Technologies, and Asensa® SC 211 sold by the company Honeywell.

b) Polar Wax

For the purposes of the present invention, the term “polar wax” is intended to mean a wax whose solubility parameter at 25° C., δa, is other than 0 (J/cm³)^{1/2}.

In particular, the term “polar wax” is intended to mean a wax whose chemical structure is formed essentially from, or even constituted of, carbon and hydrogen atoms, and comprising at least one highly electronegative heteroatom such as an oxygen, nitrogen, silicon or phosphorus atom.

The polar waxes may in particular be hydrocarbon-based, fluoro or silicone waxes.

Preferentially, the polar waxes may be hydrocarbon-based waxes.

The term “hydrocarbon-based wax” is intended to mean a wax formed essentially from, or even constituted of, carbon and hydrogen atoms, and optionally oxygen and nitrogen atoms, and that does not contain any silicon or fluorine atoms. It may contain alcohol, ester, ether, carboxylic acid, amine and/or amide groups.

According to the invention, the term “ester wax” is intended to mean a wax comprising at least one ester function. According to the invention, the term “alcohol wax” is intended to mean a wax comprising at least one alcohol function, i.e. comprising at least one free hydroxyl (OH) group.

In particular, use may be made, as polar waxes, of those chosen from:

i) waxes of formula R₁COOR₂ in which R₁ and R₂ represent linear, branched or cyclic aliphatic chains in which the number of atoms ranges from 10 to 50, which may contain a heteroatom such as O, N or P and whose melting point ranges from 25 to 120° C.;

ii) bis(1,1,1-trimethylolpropane) tetrastearate, sold under the name Hest 2T-4S® by the company Heterene;

iii) diester waxes of a dicarboxylic acid of general formula R³—(—OCO—R⁴—COO—R⁵), in which R³ and R⁵ are identical or different, preferably identical, and represent a C₄-C₃₀ alkyl group (alkyl group comprising from 4 to 30 carbon atoms) and R⁴ represents a linear or branched C₄-C₃₀ aliphatic group (alkyl group comprising from 4 to 30 carbon atoms) which may or may not comprise one or more unsaturations and which is preferably linear and unsaturated;

iv) mention may also be made of the waxes obtained by catalytic hydrogenation of animal or plant oils having linear or branched C₈-C₃₂ fatty chains, for example such as hydrogenated jojoba oil, hydrogenated sunflower oil, hydrogenated castor oil, hydrogenated coconut oil, and also the waxes obtained by hydrogenation of castor oil esterified with cetyl alcohol;

v) beeswax, synthetic beeswax, polyglycerolated beeswax, carnauba wax, candelilla wax, oxypropylenated lanolin wax, rice bran wax, ouricury wax, esparto grass wax, cork fibre wax, sugar cane wax, Japan wax, sumach wax; montan wax, orange wax, laurel wax and hydrogenated jojoba wax, sunflower wax, lemon wax, olive wax or berry wax.

According to another embodiment, the polar wax may be an alcohol wax. According to the invention, the term “alcohol wax” is intended to mean a wax comprising at least one alcohol function, i.e. comprising at least one free hydroxyl (OH) group. Alcohol waxes that may be mentioned include for example the C₃₀₋₅₀ alcohol wax Performacol® 550 Alcohol from New Phase Technologies, stearyl alcohol and cetyl alcohol.

It is also possible to use silicone waxes, which may advantageously be substituted polysiloxanes, preferably of low melting point.

The term “silicone wax” is intended to mean an oil comprising at least one silicon atom, and in particular comprising Si—O groups.

Among the commercial silicone waxes of this type, mention may be made in particular of those sold under the names Abilwax 9800, 9801 or 9810 (Goldschmidt), KF910 and KF7002 (Shin-Etsu), or 176-1118-3 and 176-11481 (General Electric).

The silicone waxes that may be used may also be alkyl or alkoxy dimethicones, and also (C₂₀-C₆₀)alkyl dimethicones, in particular (C₃₀-C₄₅)alkyl dimethicones, such as the silicone wax sold under the name SF-1642 by the company GE-Bayer Silicones or C₃₀₋₄₅ alkyl dimethylsilyl polypropylsilsesquioxane under the name SW-8005® C30 Resin Wax by the company Dow Corning.

According to one embodiment of the invention, a composition according to the invention advantageously comprises from 0% to 35% by weight, in particular from 5% to 30% by weight of wax(es), or even from 10% to 25% by weight of wax(es), relative to the total weight of the composition.

In another embodiment, a composition according to the invention comprises an amount of wax of less than 8%, or even less than 3%, and even better still less than 1% by weight, relative to the total weight of the composition.

In the context of the present invention, mention may be made, by way of particularly advantageous wax, of carnauba wax, advantageously in the form of a microdispersion, for example the one sold under the name Mexoryl SAP by the company Chimex.

Film-Forming Polymer

A composition according to the invention may preferably comprise at least an aqueous dispersion of film-forming polymer particles and optionally at least one additional film-forming polymer (not present in the form of an aqueous dispersion of particles, such as a water-soluble film-forming polymer).

In the present application, the term “film-forming polymer” is intended to mean a polymer that is capable, by itself or in the presence of an auxiliary film-forming agent, of forming a macroscopically continuous deposit, and preferably a cohesive deposit, and even better still a deposit of which the cohesion and mechanical properties are such that said deposit can be isolated and manipulated individually, for example when said deposit is prepared by pouring onto a non-stick surface such as a Teflon-coated or silicone-coated surface.

A composition according to the invention preferably comprises a total solids content of film-forming polymer(s) of greater than or equal to 5% by weight, preferably greater than or equal to 10% by weight and better still greater than or equal to 12% by weight, relative to the total weight of the composition.

A composition according to the invention preferably comprises a total solids content of film-forming polymer(s) ranging from 10% to 55% by weight, in particular from 12% to 50% by weight, relative to the total weight of the composition.

In particular, a composition according to the invention preferably comprises at least an aqueous dispersion of particles formed from one or more film-forming polymers.

It may also comprise at least one water-soluble film-forming polymer.

Thus, a composition according to the invention may comprise at least one additional film-forming polymer, different from the film-forming polymer particles present in aqueous dispersion form.

The content of this (these) “water-soluble” additional film-forming polymer(s) is preferably less than or equal to 10% by weight, even more preferentially less than or equal to 5% by weight and better still less than or equal to 2% by weight, relative to the total weight of the composition.

Film-Forming Polymer(s) in Aqueous Dispersion

Such a film-forming polymer present in said preparation of the composition in the form of particles in aqueous dispersion is generally known as a (pseudo)latex, i.e. a latex or pseudolatex. Techniques for preparing these dispersions are well known to those skilled in the art.

A dispersion that is suitable for use in the invention may comprise one or more types of particle, these particles possibly varying as regards their size, their structure and/or their chemical nature.

A composition according to the invention may comprise a total solids content of film-forming polymer particles in aqueous dispersion form of greater than or equal to 10% by weight, relative to the total weight of the composition.

Advantageously, a composition according to the invention comprises a total solids content of film-forming polymer particles in aqueous dispersion form of greater than or equal to 12% by weight, relative to the total weight of the composition.

A composition according to the invention preferably comprises a total solids content of film-forming polymer particles ranging from 10% to 55% by weight, better still from 12% to 50% by weight, relative to the total weight of the composition.

The total content of film-forming polymer particles present in aqueous dispersion form is preferably greater than or equal to 30% by weight and preferentially greater than or equal to 40% by weight, relative to the total weight of the particles.

These particles may be of anionic, cationic or neutral nature and may constitute a mixture of particles of different nature.

Among the film-forming polymers that may be used in the composition of the present invention, mention may be made of synthetic polymers, of free-radical type or of polycondensate type, and polymers of natural origin, and mixtures thereof. In general, these polymers may be random polymers, block copolymers of A-B type, multiblock copolymers A-B-A or else of ABCD type, etc, or even grafted polymers.

Free-Radical Film-Forming Polymer.

The term “free-radical polymer” is intended to mean a polymer obtained by polymerization of unsaturated and in particular ethylenically unsaturated monomers, each monomer being capable of homopolymerizing (unlike polycondensates).

The film-forming polymers of free-radical type may in particular be acrylic and/or vinyl homopolymers or copolymers.

The vinyl film-forming polymers may result from the polymerization of ethylenically unsaturated monomers containing at least one acid group and/or esters of these acid monomers and/or amides of these acid monomers.

Ethylenically unsaturated monomers bearing at least one acid group or monomer bearing an acid group that may be used include α,β-ethylenic unsaturated carboxylic acids such as acrylic acid, methacrylic acid, crotonic acid, maleic acid or itaconic acid. (Meth)acrylic acid and crotonic acid are in particular used, and more particularly (meth)acrylic acid.

The esters of acid monomers are advantageously chosen from (meth)acrylic acid esters (also known as (meth)acrylates), especially (meth)acrylates of an alkyl, in particular of a C₁-C₂₀ and more particularly C₁-C₈ alkyl, (meth)acrylates of an aryl, in particular of a C₆-C₁₀ aryl, and (meth)acrylates of a hydroxyalkyl, in particular of a C₂-C₆ hydroxyalkyl.

Among the alkyl (meth)acrylates that may be mentioned are methyl methacrylate, ethyl methacrylate, butyl methacrylate, isobutyl methacrylate, 2-ethylhexyl methacrylate and lauryl methacrylate.

Among the hydroxyalkyl (meth)acrylates that may be mentioned are hydroxyethyl acrylate, 2-hydroxypropyl acrylate, hydroxyethyl methacrylate and 2-hydroxypropyl methacrylate.

Among the aryl (meth)acrylates that may be mentioned are benzyl acrylate and phenyl acrylate.

The (meth)acrylic acid esters are in particular alkyl (meth)acrylates.

According to the present invention, the alkyl group of the esters may be either fluorinated or perfluorinated, i.e. some or all of the hydrogen atoms of the alkyl group are substituted with fluorine atoms.

Examples of amides of acid monomers that may be mentioned are (meth)acrylamides, and in particular N-alkyl(meth)acrylamides, in particular of a C₂-C₁₂ alkyl. Among the N-alkyl(meth)acrylamides that may be mentioned are N-ethylacrylamide, N-t-butylacrylamide and N-t-octylacrylamide.

The vinyl film-forming polymers may also result from the homopolymerization or copolymerization of monomers chosen from vinyl esters and styrene monomers. In particular, these monomers may be polymerized with acid monomers and/or esters thereof and/or amides thereof, such as those mentioned previously.

Examples of vinyl esters that may be mentioned are vinyl acetate, vinyl neodecanoate, vinyl pivalate, vinyl benzoate and vinyl t-butylbenzoate.

Styrene monomers that may be mentioned include styrene and α-methylstyrene.

The list of monomers given is not limiting, and it is possible to use any monomer known to those skilled in the art included in the categories of acrylic and vinyl monomers (including monomers modified with a silicone chain).

Vinyl polymers that may also be used include silicone acrylic polymers.

Mention may also be made of polymers resulting from free-radical polymerization of one or more free-radical monomers inside and/or partially at the surface of pre-existing particles of at least one polymer chosen from the group consisting of polyurethanes, polyureas, polyesters, polyesteramides and/or alkyds. These polymers are generally referred to as “hybrid polymers”.

Polycondensate:

As film-forming polymer of polycondensate type, mention may be made of anionic, cationic, nonionic or amphoteric polyurethanes, acrylic polyurethanes, polyvinylpyrrolidone-polyurethanes, polyester-polyurethanes, polyether-polyurethanes, polyureas, polyurea/polyurethanes and silicone polyurethanes, and mixtures thereof.

The film-forming polyurethane may be, for example, an aliphatic, cycloaliphatic or aromatic polyurethane, polyurea/urethane or polyurea copolymer comprising, alone or as a mixture, at least one block chosen from:
a block of aliphatic and/or cycloaliphatic and/or aromatic polyester origin, and/or
a branched or unbranched silicone block, for example polydimethylsiloxane or polymethylphenylsiloxane, and/or
a block comprising fluoro groups.

The film-forming polyurethanes as defined in the invention may also be obtained from branched or unbranched polyesters or from alkyds comprising mobile hydrogens, which are modified by reaction with a diisocyanate and a difunctional organic compound (for example dihydro, diamino or hydroxyamino), also comprising either a carboxylic acid or carboxylate group, or a sulfonic acid or sulfonate group, or alternatively a neutralizable tertiary amine group or a quaternary ammonium group.

Among the film-forming polycondensates, mention may also be made of polyesters, polyesteramides, fatty-chain polyesters, polyamides and epoxyester resins.

The polyesters may be obtained, in a known manner, by polycondensation of dicarboxylic acids with polyols, in particular diols.

The dicarboxylic acid may be aliphatic, alicyclic or aromatic. Examples of such acids that may be mentioned include: oxalic acid, malonic acid, dimethylmalonic acid, succinic acid, glutaric acid, adipic acid, pimelic acid, 2,2-dimethylglutaric acid, azelaic acid, suberic acid, sebacic acid, fumaric acid, maleic acid, itaconic acid, phthalic acid, dodecanedioic acid, 1,3-cyclohexanedicarboxylic acid, 1,4-cyclohexanedicarboxylic acid, isophthalic acid, terephthalic acid, 2,5-norboranedicarboxylic acid, diglycolic acid, thiodipropionic acid, 2,5-naphthalenedicarboxylic acid, and 2,6-naphthalenedicarboxylic acid. These dicarboxylic acid monomers may be used alone or as a combination of at least two dicarboxylic acid monomers. Among these monomers, the ones chosen in particular are phthalic acid, isophthalic acid and terephthalic acid.

The diol may be chosen from aliphatic, alicyclic and aromatic diols. The diol used is chosen in particular from: ethylene glycol, diethylene glycol, triethylene glycol, 1,3-propanediol, cyclohexanedimethanol and 4-butanediol. Other polyols that may be used are glycerol, pentaerythritol, sorbitol and trimethylolpropane.

The polyesteramides may be obtained in a manner analogous to that of the polyesters, by polycondensation of diacids with diamines or amino alcohols. Diamines that may be used are ethylenediamine, hexamethylenediamine and meta- or para-phenylenediamine. An amino alcohol that may be used is monoethanolamine.

Polymer of Natural Origin:

Use may be made in the present invention of optionally modified polymers of natural origin, such as shellac resin, sandarac gum, dammar resins, elemi gums, copal resins, water-insoluble cellulose-based polymers such as nitrocellulose, modified cellulose esters in particular including carboxyalkyl cellulose esters such as those described in patent application US 2003/185 774, and mixtures thereof.

According to a particular embodiment of the invention, said at least one film-forming polymer in the dispersed state is chosen from acrylic polymer dispersions, polyurethane dispersions, sulfopolyester dispersions, vinyl dispersions, polyvinyl acetate dispersions, vinylpyrrolidone, dimethylaminopropylmethacrylamide and lauryldimethylpropylmethacrylamidoammonium chloride terpolymer dispersions, polyurethane/polyacrylic hybrid polymer dispersions and dispersions of particles of core-shell type, and mixtures thereof.

Various types of aqueous dispersion, in particular commercial dispersions, which are suited to the preparation of the composition in accordance with the present invention are detailed below.

1/Thus, according to one preferred embodiment of the invention, the aqueous dispersion of polymer particles is an aqueous dispersion of acrylic polymer.

The acrylic polymer can be a styrene/acrylate copolymer and in particular a polymer chosen from copolymers resulting from the polymerization of at least one styrene monomer and at least one C₁-C₁₈ alkyl (meth)acrylate monomer.

As styrene monomers that may be used in the invention, examples that may be mentioned include styrene and α-methylstyrene, and in particular styrene.

The C₁-C₁₈ alkyl (meth)acrylate monomer is in particular a C₁-C₁₂ alkyl (meth)acrylate and more particularly a C₁-C₁₀ alkyl (meth) acrylate. The C₁-C₁₈ alkyl (meth)acrylate monomer may be chosen from methyl acrylate, methyl methacrylate, ethyl acrylate, propyl acrylate, butyl acrylate, butyl methacrylate, hexyl acrylate, octyl acrylate, 2-ethylhexyl acrylate, lauryl (meth)acrylate and stearyl (meth)acrylate.

As acrylic polymer in aqueous dispersion, use may be made according to the invention of the styrene/acrylate copolymer sold under the name Joncryl SCX-8211® by the company BASF or Syntran 5760cg by the company Interpolymer, the acrylic polymer sold under the reference Acronal® DS-6250 by the company BASF, or the acrylic copolymer Joncryl® 95 by the company BASF.

2/According to one embodiment variant of the invention, the aqueous dispersion of polymer particles is an aqueous dispersion of polyester-polyurethane and/or polyether-polyurethane particles, in particular in anionic form.

The anionic nature of the polyester-polyurethanes and of the polyether-polyurethanes used according to the invention is due to the presence in their constituent units of groups bearing a carboxylic acid or sulfonic acid function.

The polyester-polyurethane or polyether-polyurethane particles used according to the invention are generally sold in aqueous dispersion form.

The particle content of said dispersions currently available on the market ranges from approximately 20% to approximately 60% by weight relative to the total weight of the dispersion.

Among the anionic polyester-polyurethane dispersions that may be used in the compositions according to the invention, mention may be made in particular of the product sold under the name Avalure UR 405® by the company Noveon or Baycusan C1004 by the company Bayer Material Science.

Among the anionic polyether-polyurethane particle dispersions that may be used according to the invention, mention may be made in particular of the products sold under the name Avalure UR 450® by the company Noveon and under the name Neorez R 970® by the company DSM.

According to a particular embodiment of the invention, use may be made of a mixture of commercial dispersions consisting of anionic polyester-polyurethane particles as defined above and of anionic polyether-polyurethane particles also defined above.

For example, use may be made of a mixture consisting of the dispersion sold under the name Sancure 861® or a mixture of the product sold under the name Avalure UR 405′ and of the product sold under the name Avalure UR 450®, these dispersions being sold by the company Noveon.

3/According to another particular embodiment of the invention, the aqueous dispersion used comprises a mixture of at least two film-forming polymers in the form of particles that differ by their respective glass transition temperatures (Tg).

In particular, according to one embodiment of the invention, the composition in accordance with the invention may comprise at least a first film-forming polymer in the dispersed state and at least a second film-forming polymer in the dispersed state, said first and second polymer having different Tg values and, preferably, the Tg of the first polymer (Tg1) is higher than the Tg of the second polymer (Tg2). In particular, the difference between the Tg1 and Tg2 values is, as an absolute value, at least 10° C. and preferably at least 20° C.

More precisely, it comprises in an acceptable aqueous medium:

a) particles dispersed in the aqueous medium of a first film-forming polymer having at least one glass transition temperature Tg1 greater than or equal to 20° C., and

b) particles dispersed in the aqueous medium of a second film-forming polymer having at least one glass transition temperature Tg2 less than or equal to 70° C.

This dispersion generally results from a mixing of two aqueous dispersions of film-forming polymer.

The first film-forming polymer has at least one glass transition temperature Tg1 greater than or equal to 20° C., in particular ranging from 20° C. to 150° C. and advantageously greater than or equal to 40° C., especially ranging from 40° C. to 150° C. and in particular greater than or equal to 50° C., especially ranging from 50° C. to 150° C.

The second film-forming polymer has at least one glass transition temperature Tg2 less than or equal to 70° C., especially ranging from −120° C. to 70° C., and in particular less than 50° C., especially ranging from −60° C. to +50° C. and more particularly ranging from −30° C. to 30° C.

The measurement of the glass transition temperature (Tg) of a polymer is performed by DMTA (dynamic and mechanical temperature analysis) as described below.

To measure the glass transition temperature (Tg) of a polymer, viscoelasticity tests are performed with a “Polymer Laboratories” DMTA machine, on a sample of film.

This film is prepared by pouring the aqueous dispersion of film-forming polymer into a Teflon-coated matrix and then dried at 120° C. for 24 hours. A film is then obtained, from which specimens are cut out (for example using a punch). These specimens are typically about 150 μm thick, from 5 to 10 mm wide and have a useful length of about 10 to 15 mm. A tensile stress is imposed on this sample. The sample undergoes a static force of 0.01 N on which is superimposed a sinusoidal displacement of ±8 μm at a frequency of 1 Hz. The test is thus performed in the linear range, at low levels of deformation. This tensile stress is performed on the sample at temperatures ranging from −150° C. to +200° C., with a temperature variation of 3° C. per minute.

The complex modulus E*=E′+iE″ of the polymer tested is thus measured as a function of the temperature.

From these measurements, the dynamic moduli E′ and E″ and the damping power: tgδ=E″/E′ are deduced.

The curve of the Tgδ values is then plotted as a function of the temperature; this curve presents at least one peak. The glass transition temperature Tg of the polymer corresponds to the temperature at the top of this peak.

When the curve presents at least two peaks (in this case, the polymer has at least two Tg values), the value taken as the Tg of the polymer tested is the temperature for which the curve presents a peak of the largest amplitude (i.e. corresponding to the largest Tgδ value; in this case, only the “major” Tg is considered as the Tg value of the polymer tested).

In the present invention, the transition temperature Tg1 corresponds to the “major” Tg (in the predefined sense) of the first film-forming polymer when the latter has at least two Tg values; the glass transition temperature Tg2 corresponds to the “major” Tg of the second film-forming polymer when the latter has at least two Tg values.

The first film-forming polymer and the second film-forming polymer may be chosen, independently of each other, from free-radical polymers, polycondensates and polymers of natural origin as defined previously having the glass transition temperature characteristics defined previously.

As first film-forming polymer in aqueous dispersion, use may be made of the aqueous polymer dispersions sold under the names Neorez R-989® by the company DSM, Joncryl 95 and Joncryl® 8211 by the company BASF.

As second film-forming polymer in aqueous dispersion, use may be made, for example, of the aqueous polymer dispersions sold under the names Avalure® UR-405, Avalure® UR-460 by the company Noveon or Acrilem IC89RT® by the company ICAP, and Neocryl A-45 by the company DSM.

The film-forming polymer of the aqueous dispersion Avalure® UR-460 is a polyurethane obtained by polycondensation of polytetramethylene oxide, tetramethylxylylene diisocyanate, isophorone diisocyanate and dimethylolpropionic acid.

According to a most particularly preferred embodiment of the invention, use is made, as first and second film-forming polymers in aqueous dispersion, of the combination of styrene/acrylate polymer dispersion such as the dispersion sold under the reference Joncryl 8211® by BASF and of acrylic polymer dispersion such as the dispersion sold under the reference Neocryl A-45® by DSM.

According to another preferred embodiment, use is made, as first film-forming polymer in aqueous dispersion, of an acrylic polymer dispersion such as the dispersion sold under the reference Joncryl 95® by BASF and, as second film-forming polymer, of a dispersion of anionic polyurethane polymer sold under the reference Avalure UR405® by DSM.

As aqueous dispersions of film-forming polymer, use may be made of:
the acrylic dispersions sold under the names Acronal DS-6250® by the company BASF, Neocryl A-45®, Neocryl XK-90®, Neocryl A-1070®, Neocryl A-1090®, Neocryl BT-62®, Neocryl A-1079® and Neocryl A-523® by the company DSM, Joncryl 95® and Joncryl 8211® by the company BASF, Daitosol 5000 AD® or Daitosol 5000 SJ by the company Daito Kasey Kogyo; Syntran 5760 CG by the company Interpolymer,
the aqueous polyurethane dispersions sold under the names Neorez R-981® and Neorez R-974® by the company DSM, Avalure UR-405®, Avalure UR-410®, Avalure UR-425®, Avalure UR-450®, Sancure 875®, Avalure UR 445® and Avalure UR 450® by the company Noveon, Impranil 85® by the company Bayer, and Baycusan C1004® by the company Bayer Material Science,
the sulfopolyesters sold under the brand name Eastman AQ® by the company Eastman Chemical Products,
vinyl dispersions such as Mexomer PAM, aqueous dispersions of polyvinyl acetate such as Vinybran® from the company Nisshin Chemical or the products sold by the company Union Carbide, aqueous dispersions of vinylpyrrolidone, dimethylaminopropylmethacrylamide and lauryldimethylpropylmethacrylamidoammonium chloride terpolymer such as Styleze W® from ISP,
aqueous dispersions of polyurethane/polyacrylic hybrid polymer such as the products sold under the references Hybridur® by the company Air Products or Duromer® from National Starch,
dispersions of particles of core-shell type such as the products sold by the company Arkema under the reference Kynar® (core: fluorinated—shell: acrylic) or alternatively those described in U.S. Pat. No. 5,188,899 (core: silica—shell: silicone) and mixtures thereof.

According to a preferred embodiment, a composition in accordance with the invention comprises an aqueous dispersion of particles chosen from aqueous dispersions of acrylic film-forming polymer(s) and derivatives, in particular of styrene-acrylic and derivatives, and aqueous dispersions of polyurethane polymer(s), in particular of polyester-polyurethane, and derivatives thereof and mixtures thereof.

In the context of the present invention, as particularly advantageous film-forming polymer, mention may be made of latexes, for example the product sold under the name Syntran 5760 CG by the company Interpolymer.

According to one particular embodiment, the total content of wax(es) and the total content of film-forming polymer particles are such that the weight ratio of the wax(es) to the film-forming polymer particles is greater than or equal to 1/2 and better still 2/3.

Preferably, this ratio is inclusively between 1/2 and 2 and even more preferentially between 2/3 and 3/2.

According to one particular embodiment, the total content of wax(es) and the total content of film-forming polymer particles, which are both preferentially present in the form of particles in aqueous dispersion, with the film-forming polymer(s) chosen from aqueous dispersions of acrylic film-forming polymer(s) and derivatives, in particular styrene-acrylic and derivatives, and the aqueous dispersions of polyester-polyurethane hybrid polymer(s), and mixtures thereof, are such that the weight ratio of the wax particles to said film-forming polymer particles is greater than or equal to 1/2 and better still 2/3.

For example, this ratio is inclusively between 1/2 and 2 and even more preferentially between 2/3 and 3/2.

Preferably, the particles in the dispersed state may be chosen from a wax, latex, and mixtures thereof.

According to one particular embodiment variant, a composition according to the invention consists of a particulate dispersion comprising at least one mixture of a microdispersion of carnauba and a latex.

Colorant

The compositions in accordance with the invention preferably comprise at least one colorant.

This (or these) colorant(s) is (or are) preferably chosen from pulverulent materials, liposoluble dyes and water-soluble dyes, and mixtures thereof.

Preferably, the compositions according to the invention comprise at least one pulverulent colorant. The pulverulent colorants may be chosen from pigments and nacres, and preferably from pigments.

The pigments may be white or coloured, inorganic and/or organic, and coated or uncoated. Among the inorganic pigments, mention may be made of metal oxides, in particular titanium dioxide, optionally surface-treated, zirconium, zinc or cerium oxide, and also iron, titanium or chromium oxide, manganese violet, ultramarine blue, chromium hydrate and ferric blue. Among the organic pigments that may be mentioned are carbon black, pigments of D&C type and lakes based on cochineal carmine or on barium, strontium, calcium or aluminium.

The nacres may be chosen from white nacreous pigments such as mica coated with titanium or with bismuth oxychloride, coloured nacreous pigments such as titanium mica with iron oxides, titanium mica with in particular ferric blue or chromium oxide, titanium mica with an organic pigment of the abovementioned type, and also nacreous pigments based on bismuth oxychloride. Examples of nacres that may also be mentioned include natural mica coated with titanium oxide, with iron oxide, with natural pigment or with bismuth oxychloride.

Among the commercially available nacres that may be mentioned are the nacres Timica, Flamenco and Duochrome (on mica base) sold by the company Engelhard, the Timiron nacres sold by the company Merck, the Prestige nacres on mica base sold by the company Eckart and the Sunshine nacres on synthetic mica base sold by the company Sun Chemical.

The nacres may more particularly have a yellow, pink, red, bronze, orange, brown, gold and/or coppery colour or tint.

The liposoluble dyes are, for example, Sudan Red, D&C Red 17, D&C Green 6, β-carotene, soybean oil, Sudan Brown, D&C Yellow 11, D&C Violet 2, D&C Orange 5, quinoline yellow and annatto.

Preferably, the pigments contained in the compositions according to the invention are chosen from metal oxides.

A composition according to the invention may also comprise at least one particulate or non-particulate, water-soluble or water-insoluble colorant, preferably in a proportion of at least 0.01% by weight relative to the total weight of the composition.

For obvious reasons, this amount is liable to vary significantly with regard to the intensity of the desired colour effect and of the colour intensity afforded by the colorants under consideration, and its adjustment clearly falls within the competences of those skilled in the art.

These colorants may be present in a content ranging from 0.01% to 30% by weight relative to the total weight of the composition and in particular from 6% to 22% by weight relative to the total weight of the composition.

Preferably, the colorant(s) is (are) chosen from one or more metal oxide(s) present in a content of greater than or equal to 2% by weight relative to the total weight of the composition, and advantageously inclusively between 6% and 22% by weight relative to the total weight of the composition.

Device

As previously mentioned, a cosmetic assembly according to the invention contains, in addition to a cosmetic composition, a device comprising a mould, the mould comprising at least one cavity, and preferably several cavities, into which or each of which at least one of said fibres can be at least partially introduced, for the purposes of moulding the composition onto at least one part of said fibre(s) present in the cavity or cavities.

Mould

The moulding is carried out in situ in the mould cavity or cavities, and allows the composition to be shaped. Thus, the composition can fit the shape of the mould cavity or cavities.

The mould may comprise one or more imprints, which each at least partially define a mould cavity. Preferably, the mould is not limited to a single cavity. In particular, the mould comprises at least two cavities, preferably at least six cavities, and even more preferentially at least ten cavities.

The mould may comprise at least two parts which each comprise one or more imprints, the imprint(s) of one of the parts being placed opposite the imprint(s) of at least one other part, so as to form the mould cavity or cavities when the parts of the mould are brought together, each of the imprints being in particular of rounded cross section, notably of circular arc, elliptical arc or parabolic cross section, in particular of semicircular cross section.

The mould may comprise at least two parts, one part comprising one or more imprints, each of the imprints being in particular of rounded cross section, notably of circular arc, elliptical arc or parabolic cross section, in particular of semicircular cross section, the other part being devoid of imprint, so as to form the mould cavity or cavities when the parts of the mould are brought together. Such a mould has the advantage of not requiring a great deal of precision when placing the two parts of the mould opposite one another so as to form the cavities.

The imprint(s) may be of rounded cross section to fit the keratin fibre(s) shape.

The imprints may be parallel to one another.

The imprints may be arranged along only one part of the mould, in particular in the front of said mould, i.e. on the side where the keratin fibres are introduced.

Each imprint may have a depth of between 75 μm and 1.5 mm.

The mould may remain motionless relative to the keratin fibres during the moulding of the fibres in its cavity or cavities.

The mould preferably does not move along the keratin fibres during the moulding of the fibres in its cavity or cavities.

The mould may or may not be disposable. In particular, it is disposable.

The mould, in particular if it is disposable, may comprise an absorber specific to the liquid medium.

The mould may comprise an absorbent material or be covered with absorbent or desiccating material, in particular a ceramic or a porous material, or a chemical active agent which is capable of absorbing, such as a silica gel or at least one compound such as an aerogel, clay, bentonite, calcium or cobalt or copper salts (chlorate, sulfate), lithium halides, potassium carbonate, magnesium sulfate or sodium sulfate, or organic compounds such as sugars.

The surface of the mould may be smooth so as to confer on the composition once moulded a smooth external surface and a glossy appearance.

There can not be any relative speed between the keratin fibres and the cavities during the moulding, for example during a period required for the hardening or the drying of the composition, for example a period of typically 10 to 60 seconds.

Mould Cavity

The volume of the cavity or cavities may exceed by at least a factor of 2, better still a factor of 10 to 100, the volume of the part of the keratin fibres filling it or them.

The volume of each cavity may range between 0.08 and 220 mm³.

The cavity or cavities may each have a circular, semicircular, oval or polygonal cross section, which may or may not be constant when moving along the cavity.

The cavity or cavities may each be approximately cylindrical in shape.

The cavity or cavities may each have a decreasing cross section so as to give the fibre(s), once moulded by the composition, a tapered appearance and to facilitate, where appropriate, the demoulding of the fibres by pulling them away.

The cavity or cavities may each have an approximately cylindrical shape on one part of their length and an enlarged, in particular spherical, shape on another part of their length, in particular with a view to moulding a head, for example in the shape of a portion of a sphere, on at least one part of the keratin fibre(s).

The cavity or cavities may each have a length of between 5 and 30 mm and a width, in particular a diameter, of between 150 μm and 3 mm. In the case of a length of 30 mm and a diameter of 3 mm, the volume may be increased by a factor of 50.

Each cavity may have an elongated shape with a curvilinear longitudinal axis which approximately reproduces or which increases the curvature of the keratin fibre(s) introduced, in particular the eyelashes. Thus, the moulding enables to increase keratin fibres visibility while underlining their curvature.

The radius of curvature of the cavity or cavities may be higher than 15 mm. In particular, it can range between 15 and 25 mm.

Each cavity may, on the contrary, have an elongated shape which does not reproduce the curvature of the keratin fibre(s) introduced, having in particular a rectilinear or broken-line longitudinal axis. The cavity or cavities may extend in all directions, in particular transversely to the eyelashes. The cavity or cavities may form patterns of any types, such as waves or grids.

Each cavity may have an elongated shape, with a curvilinear or rectilinear longitudinal axis, and the cavities may have non-parallel respective longitudinal axes.

When the cavity or cavities have an elongated shape, the keratin fibres are preferably introduced into the cavity or cavities along their longitudinal axes.

Although cavities of elongated shape make it possible to perform a more conventional cosmetic treatment of keratin fibres, new effects can be obtained with cavities of non-elongated shape. For example, the cavity or cavities may each have an approximately spherical or polyhedral shape, in particular spherical shape, having a diameter of between 0.4 mm and 5 mm, so as to form balls at the end of the keratin fibres, for example.

Each cavity is preferably closed laterally, so that the composition does not escape laterally during the moulding.

Each cavity may have an elongated shape and be closed at least one of its longitudinal ends.

Each cavity may constitute a space which is substantially entirely closed, notably entirely closed, in the absence of keratin fibre. In this case, one part of each keratin fibre at least partially introduced into a cavity may be gripped between the two parts of the mould that are brought together, while another part of the keratin fibre extends into the cavity. The cavity may be closed on the side where the keratin fibre(s) is (are) introduced, via a flexible wall, which can deform, preferably elastically, so as to allow the keratin fibre(s) to pass into the cavity.

As a variant, each cavity may constitute a semi-open space, in the absence of keratin fibre. In particular, each cavity can delimit a space which is closed with the exception of one end via which the keratin fibre(s) which is (are) at least partially introduced into the cavity communicate(s) with the exterior. Thus, each cavity may open to the exterior, on the side where the keratin fibre(s) is (are) introduced, via an opening through which the keratin fibre(s) is (are) introduced.

The free end of the keratin fibre(s) introduced into the cavity or cavities preferably do not exceed the cavity or cavities.

At least two of the cavities may be not connected. All of the cavities may be not connected.

The cavities may or may not be parallel to one another.

The cavities may be placed at regular intervals, for example according to a step of 1 to 2 mm, centre to centre.

The number of cavities may be greater than or equal to one, in particular greater than or equal to two, preferably greater than or equal to six and more preferably greater than or equal to ten.

Jaws

The cavity or cavities may be formed by bringing two jaws together, in particular according to a translational and/or rotational movement of one of the jaws with respect to the other.

The two jaws can be moved apart at the moment the keratin fibres are introduced and closed again so as to form the cavities into which the keratin fibres are introduced.

The jaws may carry or integrate the mould.

The jaws may be curved, in particular about a geometric axis perpendicular to the longitudinal axis of the mould cavities.

A system where one or both of the jaws slide(s) on columns can be used to move the two jaws apart at the moment the keratin fibres are introduced and to close them again so as to form the cavities.

Use may also be made of a system where the jaws are connected by a hinge.

Use may also be made of a system where the jaws are not connected, and are optionally associated with one another by a guide system.

The two jaws preferably fit together sufficiently well for the composition present in the cavities not to escape.

The jaws may comprise an elastomer material, for example in order to absorb a deformation of the mould.

One or both of the jaws may have a system for admitting drying compounds, such as a calcium salt for example.

Mould Deformation

It may be advantageous to deform the mould with the keratin fibres in place in order to reduce the volume of the cavities, and for example to force the composition to be distributed around the fibres introduced into the cavities.

The mould may comprise a flexible, in particular elastomeric, material.

An extensible, in particular elastomeric, mould makes it possible to compress the composition in order to avoid problems of dead volume and/or to force better integration of the fibres into the composition. The mould may be entirely made of flexible, in particular elastomeric, material.

The mould may comprise flexible parts and non-flexible parts. The mould may in particular be surface-covered with flexible, in particular elastomeric, material, for example over a thickness ranging from 0.5 to 2 mm.

The deformation of the mould may be carried out by compression, for example mechanical compression with fingers, or be pneumatic or hydraulic, or by suction. For example, by pulling a trigger, an overpressure can be created between one jaw and the mould, which has the effect of compressing the two parts of the mould on to one another and preventing dead zones.

Demoulding

The composition can be demoulded, and extracted from the mould, preferably without losing its cohesion around the keratin fibres and while retaining the surface finish conferred by the mould.

Demoulding of the composition without deterioration is desired.

The mould may be extensible, in particular elastomeric, in order to facilitate demoulding.

The mould cavity or cavities may have a non-stick coating or may have undergone a treatment aimed at conferring non-stick properties. Thus, the mould may comprise at the surface a low-adhesion material, in particular of silicone or PTFE type. A layer of a non-stick product, in particular an oil, a silicone, a PTFE powder or boron nitride, may also be applied to the mould cavity.

The device may comprise an automatic or non-automatic demoulding system in order to act on the mould and/or the composition contained in the cavity or cavities in order to facilitate the separation of the composition-coated fibres from the mould. This demoulding system may comprise a set of small blades or other reliefs which deform the mould by being compressed against said mould. The deformation of the mould can take place at the level of the cavities, thereby facilitating the ejection of the moulded material.

Bridges of composition may connect several cavities to one another after moulding, in an unwanted manner. The device may comprise blades or other reliefs which are used to cut bridges of composition between at least two cavities after moulding. These blades or other reliefs may act by fitting over the bridges between the cavities. These blades may be added to the mould.

The blades or other reliefs which are used to cut the bridges of composition may be carried by one of the jaws. In this case, the other jaw may have a planar surface from the viewpoint of the blade or other relief or a groove into which the blade or other relief fits. The blades or other reliefs may further be placed on the two jaws, in such a way that one blade or other relief of one of the jaws fits over one blade or other relief of the other jaw.

The demoulding of the moulded composition is preferably carried out mechanically, in particular by deformation of the mould, by moving the two jaws away from one another and/or by pulling the keratin fibres out of the mould.

The demoulding of the composition may be carried out, where appropriate, by pulling at one end of one part of the mould in order to separate it from the other part, in the way in which two sheets adhering to one another are separated by peeling. The demoulding of the moulded composition may further, according to one variant, be at least partly carried out physicochemically, in particular by at least partially dissolving, with a solvent, the mould or a film-coating present inside the mould, between said mould and the composition moulded onto the keratin fibres.

Provision of the Composition

At least one part of the composition, in particular the entire composition, may be deposited on at least one of said fibres, better still on each of said fibres, before they are introduced into the mould. In this case, the closing of the mould can distribute the composition in the cavities and, where appropriate, drive the excess composition out of the mould.

At least one part of the cosmetic composition, in particular the entire cosmetic composition, may be initially present in the mould before introduction of the fibre(s) into the mould. This can facilitate the metering of the composition introduced into the mould cavity or cavities. The composition may be contained with the mould in sealed packaging.

At least one part of the composition, in particular the entire composition, may be injected into the mould, via at least one injection channel.

The mould may comprise imprints in which at least one part of the composition, in particular the entire composition, is present before introduction of the keratin fibre(s) into the mould. The imprints define the mould cavities when said mould is closed.

The mould may comprise at least two parts each comprising imprints, at least one part of the composition, in particular the entire composition, being present in at least one part, in particular in all the parts, of the mould, before introduction of the keratin fibre(s) into the mould. The composition for example entirely fills the imprints and is present only in the imprints, before closing of the mould.

The mould may also be prefilled with an excess of the composition so that the material stands higher than the cavity, for example by being slightly curved. In this way, any absence of product around the eyelash is avoided and the formation of a perfect cast is ensured.

Automatism

The device may be automated. A single triggering may make it possible to perform a series of operations, for example filling the cavities with the moulding composition, and an action exerted on the mould so as to deform it in order to perform demoulding. The closing of the mould may also be automatic.

Device

The device may comprise at least one heating element which serves to increase the temperature of the cosmetic composition, the increase in the temperature inducing solidification of the composition.

During the temperature increase, the composition may be present in the mould or outside the mould. The composition may be present outside the mould during the increase in its temperature and may be injected into the mould, in particular via at least one injection channel.

The heating may be automatically triggered upon closure of the mould. As a variant, the heating may be triggered before closure of the mould and a visual and/or sound indicator may signal to the user that the desired temperature for closing the mould and/or putting the fibres in place has been reached.

The device may comprise at least one evaporation system which serves to evaporate the liquid medium. This may be a heating element and/or a suitable ventilation.

The device may comprise at least one system which assists the drying of the cosmetic composition, the system providing energy, in particular in the form of light or heat, and/or comprising an aeration circuit, suction and/or blowing of air.

The cavity or cavities of the mould can be formed by bringing two jaws together, the jaws remaining closed until the composition has solidified.

The device may comprise at least one system for admitting material, which serves to introduce a part or all of the cosmetic composition into the mould cavity or cavities, and in particular comprises one or more injection channels communicating with one or more mould cavities. The composition is, for example, contained in a reservoir, and a piston or a pump makes it possible to force it to flow into the mould cavity or cavities.

The device may comprise a heating element which is at least one light element, in particular IR, UV or visible light element, or microwave element, which serves to increase the temperature of the composition, in particular by absorption of the light or microwave radiation by the cosmetic composition.

The jaws may be closed manually.

The device may be in the form of a clamp comprising at least one housing into which at least one of the fingers of one hand, for example the thumb or the index finger, can be introduced so as to move the two jaws apart, in order to make it possible to introduce the keratin fibres and to close them on said fibres.

The device may be in the form of a clamp comprising two housings into which two of the fingers of one hand, for example the thumb and the index finger, can be introduced so as to move the two jaws apart, in order to make it possible to introduce the keratin fibres and to close them on said fibres. Each of these housings may be removable or non-removable. It may or may not be possible for each of these housings to be oriented rotationally.

Cosmetic Treatment Process

As previously mentioned, according to another of its aspects, the present invention also relates to a process for cosmetic treatment of one or more keratin fibres, in particular of one or more eyelashes or eyebrows, comprising at least the step consisting in moulding a fluid cosmetic composition, containing at least particles in the dispersed state in a liquid medium, said composition being solidifiable via the aggregation of said particles, onto at least one part of said fibres, by means of one or more cavities (5), of a mould (2), into which said fibres are at least partially introduced.

In particular, said fibres are, optionally individually, at least partially introduced into said cavities.

The composition may be deposited on at least three quarters of the length of at least one of said fibres, better still on at least three quarters of the length of each of said fibres.

The length of a fibre is measured from the surface of the skin up to its free end when the fibre is placed flat.

The composition may be deposited on just one part of the length of at least one of said fibres, better still on just one part of the length of each of said fibres.

Preferably, the cosmetic composition is introduced in the liquid state into the mould so as to subsequently solidify therein, in particular via the aggregation of the particles dispersed in the liquid medium, the aggregation being in particular induced by partial or total evaporation of said liquid medium.

According to one particular embodiment, the cosmetic composition is moulded onto at least one part of at least one of said keratin fibres and of at least one additional fibre, the cosmetic composition ensuring the attachment of the additional fibre(s) to the keratin fibre(s), in particular with the additional fibre(s) dedicated to extending the keratin fibre(s), with or without axial overlap between the two types of fibres.

Keratin Fibres

The keratin fibres to which the treatment according to the invention applies are preferably human, in particular eyelashes or eyebrows, more preferably eyelashes.

The keratin fibres may be hair. It is thus possible to treat the hair, in particular on a part of the length thereof, for instance the roots, in order to increase the rigidity thereof, and/or the ends in order to improve the appearance thereof.

It is possible to post-treat the keratin fibres, moulded according to the invention, with other products, for example mascara.

Each of said fibres may be at least partially introduced into a respective cavity. At least two of said fibres may be at least partially introduced into the same cavity. At least one cavity may contain just one fibre.

The keratin fibres may be covered with an absorbent material as previously described, before they are introduced into the mould.

Additional Fibres

The assembly and the process according to the invention also make it possible to attach additional fibres to existing keratin fibres via the composition while limiting the visibility of the connection, thereby making it possible to give the keratin fibres a longer and/or thicker appearance.

Thus, the composition may be moulded onto at least one part of at least one of said keratin fibres and of at least one additional fibre. Thus, the device may comprise one or more additional fibres ensuring the attachment of the additional fibre(s) to the keratin fibre(s), in particular with the additional fibre(s) dedicated to extending the keratin fibre(s), with or without axial overlap between the two types of fibres

The composition may be deposited on just one part of the length of at least one of the keratin fibres and on just one part of the length of at least one additional fibre, better still on just one part of the length of each of the keratin fibres and on just one part of the length of each of the additional fibres.

The additional fibres may be covered with the composition only at their ends directed towards the keratin fibres, preferably over a length of less than 10 millimetres, and preferably less than 5 mm.

The device may comprise one or more of said additional fibres, prepositioned in the mould cavity or cavities or interlinked with a support which makes it possible to preposition them. This support may be removable or resorbable, for example by washing with water.

The additional fibres may be synthetic fibres of the same colour as the keratin fibres of the user, and in particular may be darker in colour than the eyelashes of the user and in particular black. Typically, the fibres are 50 μm to 200 μm in diameter, and between 5 mm and 25 mm in length, and can be tapered in order to resemble natural eyelashes.

At least one part of the composition, in particular the entire composition, may be deposited on the additional fibre(s) before they are introduced into the mould.

At least one part of the composition, in particular the entire composition, may be deposited on the additional fibre(s) while said fibres are present in the mould.

At the least one part of the composition, in particular the entire composition, may be initially present in the mould before introduction of the additional fibres into the mould.

At least one part of the composition, in particular the entire composition, may be injected into the mould, via at least one injection channel.

Heat then makes it possible to attach the additional fibres to the keratin fibres.

The device may comprise two jaws, at least one of the jaws comprising a heating element.

The device may comprise a light source and two jaws, at least one of the two jaws being transparent to at least a part of the radiation emitted by the light source, such that said radiation reaches the composition.

The keratin fibres may be cut prior to their introduction into the mould, in particular in order to give them a length which will make them fit to the arrangement of the additional fibres in the mould.

Other features, advantages and methods of application of the particles and of the preparation process according to the invention will emerge more clearly from the exemplary embodiments of the invention and from the examination of the appended figures, presented by way of non-limiting illustration of the field of the invention and in which:

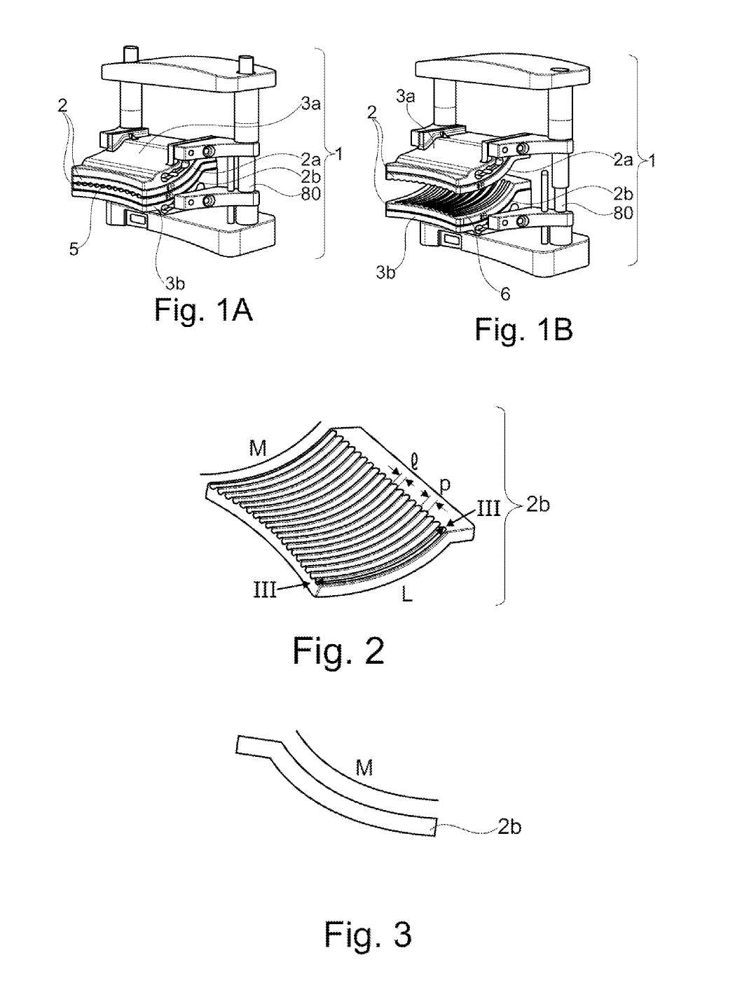

FIGS. 1A and 1B represent an example of an assembly according to the invention, respectively with the jaws of the mould closed in moulding configuration and open;

FIG. 2 represents in isolation one of the parts of the mould of FIGS. 1A and 1B;

FIG. 3 represents, in section along III of FIG. 2, a part of the mould of this figure;

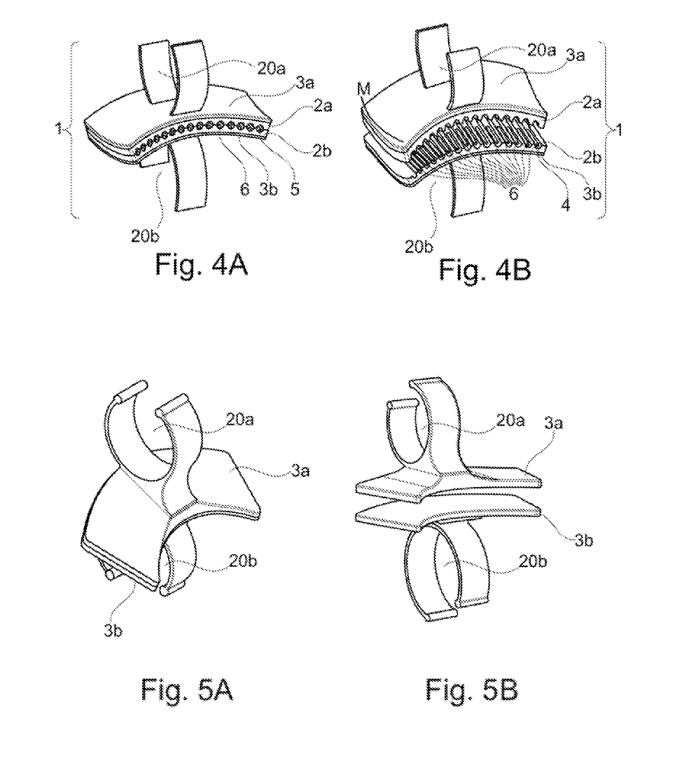

FIGS. 4A and 4B represent another example of an assembly according to the invention, in the form of a clamp, respectively with the jaws of the mould closed in moulding configuration and open;

FIGS. 5A and 5B, 7A and 7B, 9 and 10 represent other examples of devices according to the invention in the form of a clamp, the moulds not being represented, FIGS. 5A and 5B illustrating the same example of a device respectively with the jaws of the mould closed and open, FIGS. 7A and 7B illustrating the same example of a device respectively with the jaws of the mould closed and open;

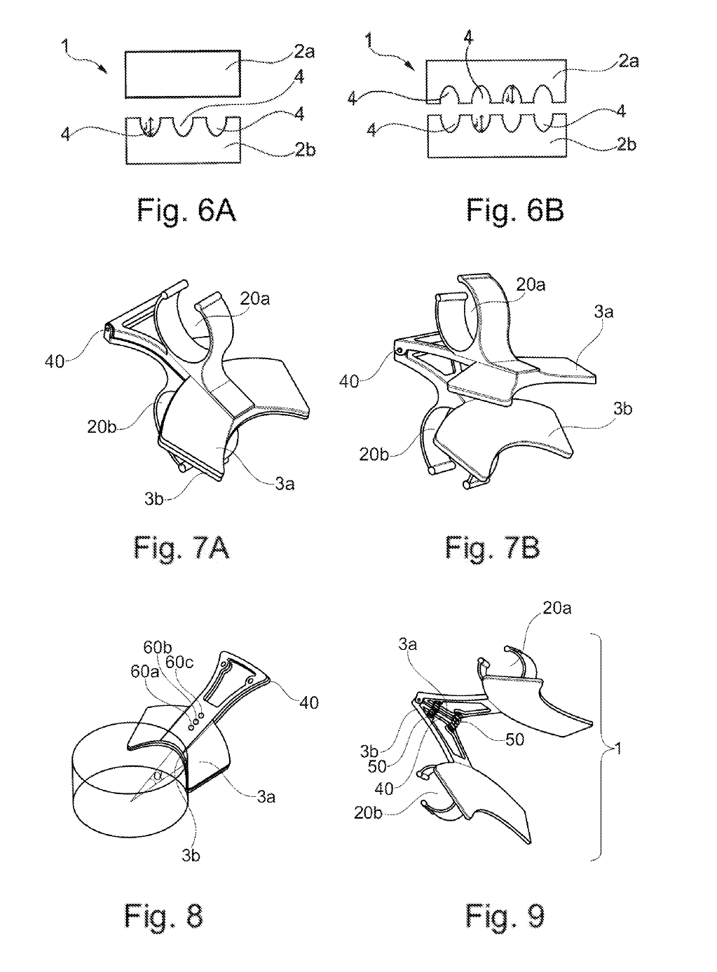

FIGS. 6A and 6B represent two examples of a mould before closing;

FIG. 8 represents the device from FIG. 7A, the housings having been dismantled;

FIGS. 11AB and 11B represent various steps for using an example of a demoulding system;

FIG. 12 represents another example of a mould before closing;

FIG. 13 represents an example of a part of a device equipped with blades;

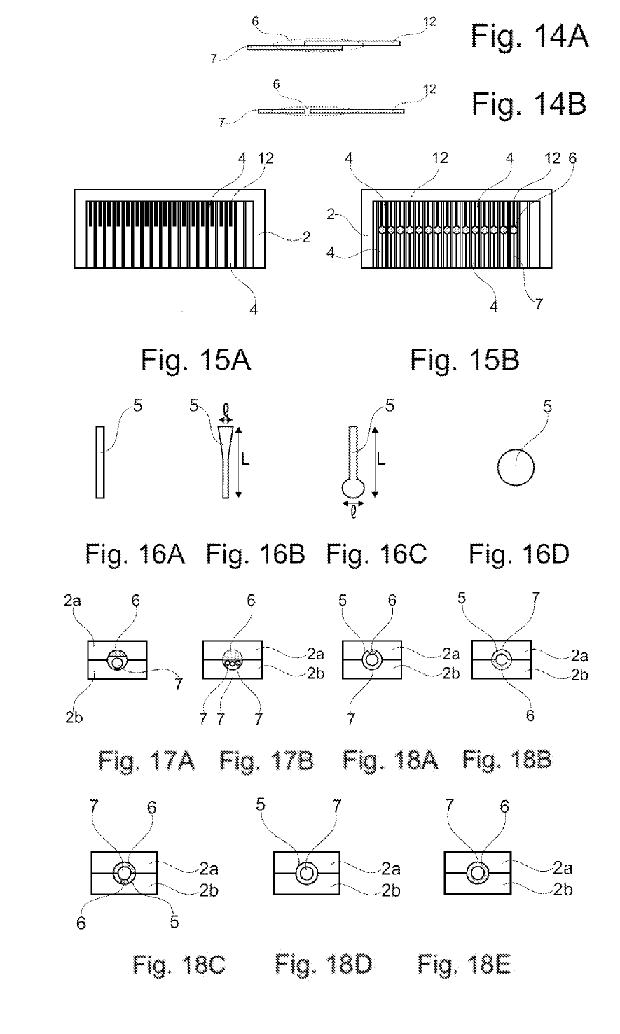

FIGS. 14A and 14B represent two examples of connection of an additional fibre to an eyelash using a composition according to the invention;

FIGS. 15A and 15B illustrate an example of attachment of additional fibres to eyelashes;

FIGS. 16A to 16D represent, in section, examples of mould cavity shape;

FIGS. 17A and 17B represent respectively a cavity comprising an eyelash and a cavity comprising several eyelashes;

FIGS. 18A to 18E represent, in section, examples of arrangements of the composition according to the invention before moulding, within a mould cavity into which an eyelash is introduced;

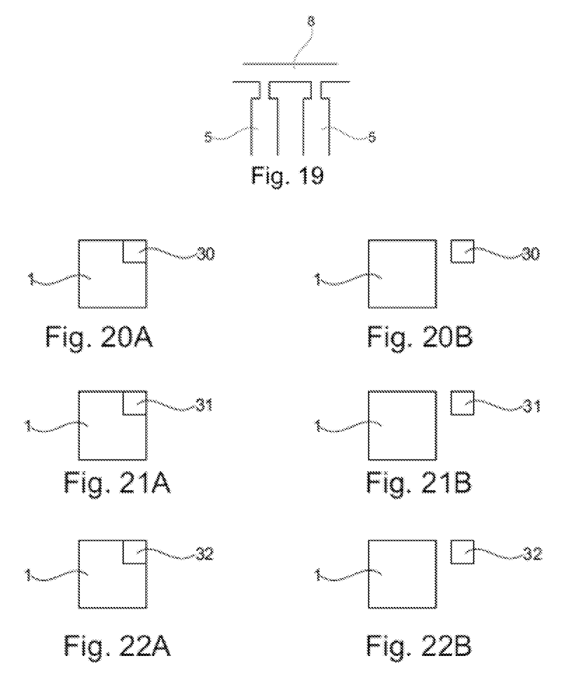

FIG. 19 represents an example of an injection channel communicating with mould cavities;

FIGS. 20A and 20B represent, very diagrammatically, respectively a device according to the invention comprising a heating element and a device according to the invention placed in proximity to a heating element;

FIGS. 21A and 21B represent, very diagrammatically, respectively a device according to the invention comprising a system which assists drying and a device according to the invention placed in proximity to a system which assists drying;

FIGS. 22A and 22B represent, very diagrammatically, respectively a device according to the invention comprising a light element and a device according to the invention placed in proximity to a light element;

FIGS. 1A and 1B represent an example of an assembly according to the invention comprising a device (1) and a cosmetic composition (6) for cosmetic treatment of eyelashes.

The device (1) comprises a mould (2) which has two parts (2a) and (2b). The mould (2) preferably comprises an elastomeric material. The two parts (2a) and (2b) of the mould (2) each have a thickness e of approximately 2 mm for example.

The two parts (2a) and (2b) of the mould (2) comprise imprints (4), in the form of grooves of elongated shape. Each part (2a, 2b) comprises, for example, as illustrated, 17 imprints (4), for example of width l of approximately 0.5 mm, of length L of approximately 2.5 cm and of approximately semicircular cross section. The imprints (4) of each part (2a, 2b) of the mould (2) are preferably substantially parallel to one another. The imprints are also, preferably as illustrated, evenly spaced out, according to a step p of 1.5 mm, centre to centre.

The device (1) comprises two jaws (3a) and (3b) which carry respectively the mould parts (2a) and (2b).

The two parts of the mould (2) and the jaws (3a) and (3b) may have additional reliefs, so as to be able to accurately place the two parts (2a, 2b) of the mould (2) on each jaw (3a, 3b).

The lower jaw (3b) comprises, in the example under consideration, a temperature-controlled heating element (30).

The lower jaw (3b) is fixed, whereas the upper jaw (3a) slides vertically by means of a guidance system (80).

When the jaws (3a) and (3b) are closed against one another, as represented in FIG. 1A, cavities (5) are formed by virtue of the imprints (4), which are superimposed in pairs.

In the example under consideration, 17 cavities of elongated shape, having a width l of approximately 0.5 mm, a length L of approximately 2.5 cm and an approximately circular cross section, which are substantially parallel to one another and evenly spaced out, according to a step p of 1.5 mm, centre to centre, are thus created.

The cavities (5) are closed on the sides and open out to the exterior, on the side where the eyelashes are introduced, via a front opening through which the eyelashes are introduced. The cavities are closed at their rear end.

To use the device (1), the cosmetic composition (6) is, for example, placed in imprints (4) of the part (2b) of the mould (2), mounted on the lower jaw, before introduction of the eyelashes. The composition (6) is a composition as previously described.

The eyelashes are introduced into the imprints (4) of the mould (2) of the device (1) in its open form (FIG. 1B), then the jaws (3a, 3b) are closed so as to hold the eyelashes in the cavities (5).

The heating element (30) brings the composition (6) to a temperature above 45° C., such that the composition (6) solidifies via the reorganization, or even the aggregation, of the particles dispersed in the liquid medium, and that it takes the shape of the imprints (4). The moulding of the composition onto the eyelashes is carried out in situ in the cavities (5) of the mould (2).

Then, the heating element (30) stops heating the composition and said composition is left to cool.

The jaws (3a, 3b) are opened and the eyelashes are withdrawn when the temperature of the composition (6) has come back down. The device (1) can be produced such that the closing of the jaws (3a) and (3b) automatically triggers the heating of the composition (6), and so that the heating is automatically stopped when the desired temperature is reached.

The opening of the jaws (3a) and (3b) can take place automatically, as appropriate. As a variant, a sound or light signal can indicate that the jaws can be opened.

FIGS. 2 and 3 represent more particularly the part (2b) of the mould (2) of FIGS. 1A and 1B.

It is seen that the part (2b) can be curved along the longitudinal direction M of the imprints (4), in order to reproduce the curved shape of the eyelashes, the radius of curvature preferably being between 15 and 25 mm.

The edge of the part (2b) intended to come into contact with the eyelid may have a rounded shape, concave towards the exterior, with a radius of curvature of preferably between 15 and 25 mm.

The device (1) may, according to one implementation variant of the invention, be in the form of a clamp, as represented in FIGS. 4A and 4B.

Like the device represented in FIGS. 1A and 1B, this device in the form of a clamp comprises a mould (2) in two parts (2a) and (2b). The mould (2) preferably comprises an elastomeric material. The two parts (2a) and (2b) of the mould (2) each have, for example, a thickness e of approximately 2 mm. The two parts (2a) and (2b) of the mould (2) comprise imprints (4), in the form of grooves of elongated shape. The device (1) comprises two jaws (3a) and (3b) which carry respectively the mould parts (2a) and (2b).

Each part (2a, 2b) comprises, for example, 15 imprints (4), for example having a width 1 of approximately 0.5 mm, a length L of approximately 2.5 cm, and a decreasing semicircular cross section so as to give the eyelashes, once moulded with the composition (6), a tapered appearance. The imprints (4) of each part (2a, 2b) of the mould (2) are preferably, as illustrated, substantially parallel to one another. The imprints are evenly spaced out, according to a step p of 1.5 mm, centre to centre.

The mould (2a, 2b) may have a radius of curvature along the direction M of FIG. 4B of between 15 and 25 mm in order to follow the shape of the eyelashes. The edge of the part (2b) intended to come into contact with the eyelid may have a rounded shape, concave towards the exterior, with a radius of curvature of preferably between 15 and 25 mm.

The edge of the jaws (3a, 3b) and of the mould (2a, 2b) intended to come into contact with the eyelid may have a rounded shape, concave towards the exterior, in particular circular, the edge of the eyelid describing, to a first approximation, an arc of a circle.

When the jaws are closed against one another, as represented in FIG. 4A, cavities (5) are formed by virtue of the imprints (4), which are superimposed in pairs. 15 cavities (5) of elongated shape, having a width 1 of approximately 0.5 mm, a length L of approximately 2.5 cm and a circular cross section of decreasing diameter as the distance from the opening for introducing the eyelashes increases, which are substantially parallel to one another and evenly spaced out, according to a step p of 1.5 mm, centre to centre, are thus, for example, created. The cavities (5) are closed on the sides and open out to the exterior, on the front side where the eyelashes are introduced, via a circular opening through which the eyelashes are introduced. The cavities are closed at their rear end.

The device (1) in the form of a clamp may comprise, as illustrated, two housings (20a, 20b) into which fingers of one hand, for example the thumb and the index finger, can be introduced so as to move the two jaws (3a, 3b) apart, in order to make it possible to introduce the eyelashes between them and to close them on said eyelashes.

The cosmetic composition (6) to be moulded is placed in imprints (4) of the part (2b) of the mould (2) before introduction of the eyelashes. The composition (6) is a composition as previously described.

An independent oven, not represented, can be used to bring the composition to the desired temperature, before introduction of the eyelashes into the imprints (4) of the mould. The housings (20a, 20b) preferably remain cold, the heating preferably taking place by radiative transfer on the faces of the mould having the imprints.

The cavity of the oven may have a parallelepipedal shape, for example an approximately 10 cm-sided parallelepipedal shape. In addition to its heating function, the oven can also make it possible to present the jaws in such a way that the user can seize them directly between the thumb and index finger, in a single action. In particular, it is possible to use portable clamps of which the internal faces are placed in front of the heat sources and the external faces bear grasping rings adjusted to the shape of the fingers.

The surfaces carrying the composition can, for example, be heated in the range 50° C.-80° C. The oven can be powered by a low-voltage power source.

The heating temperature can be adjustable by the user. A casing can enable a set temperature to be displayed. The heating power can be about 5 W for example.

When the composition is at the desired temperature, the user's thumb and index finger are introduced into the housings (20a and 20b), then the device (1) is brought into proximity to the eyelashes and the latter are introduced into the imprints (4) of the mould (2) of the device (1) in its open form (FIG. 4B), then the jaws (3a, 3b) are closed so as to hold the eyelashes in the cavities (5).

The moulding is carried out in situ in the cavity or cavities (5) of the mould (2).

Next, the jaws (3a, 3b) are opened and the eyelashes are withdrawn when the temperature of the composition (6) has come back down.

The device (1) in the form of a clamp may be devoid of a system for guiding the movement of one jaw relative to the other, as represented in FIGS. 5A and 5B. This device has two housings (20a, 20b) for the fingers, like the example previously described.

Only one of the parts can have imprints, which is particularly advantageous for a device (1) devoid of a guidance system, since the bringing together of the two parts of the mould does not need to be carried out very accurately.

By way of example, represented in FIG. 6A is a device (1) in which the part (2a) of the mould (2) may be smooth and the part (2b) may comprise semicircular imprints (4).

Moreover, each cavity (5) may have an elongated shape which does not reproduce the curvature of the eyelash(es) introduced, it being possible in particular for the cavity to be non-curved along its longitudinal direction, having, for example, a rectilinear longitudinal axis.

The device (1) in the form of a clamp may comprise a guidance system, for example in the form of reliefs having complementary shapes, which brings the jaws (3a, 3b) into position so that the two parts of the mould (2a, 2b) accurately correspond to one another. The device (1) in the form of a clamp may thus comprise, for example, a male and female cone guidance system.

The device (1) in the form of a clamp may comprise a guidance system of film hinge type or other form of articulation (40), as represented in FIGS. 7A and 7B, such that the jaws can be moved together by a pivoting movement with respect to one another.

Each cavity formed by the superimposition of two imprints may have a rectilinear longitudinal axis. The front edges of the jaws (3a, 3b) intended to come into contact with the eyelid may have a circular shape, as illustrated.

During the use of the device (1), the angle α made by the plane of moulding with the horizontal may be zero or non-zero, in particular between 20° and 40°, as illustrated in FIG. 8. Inclining the plane of the moulds makes it possible to fix the curvature of the edge coming into contact with the eyelid as close as possible to the curvature of the eyelid itself.

The hinge (40) may comprise one or more springs (50) as represented in FIG. 9, which assist the opening movement for example.

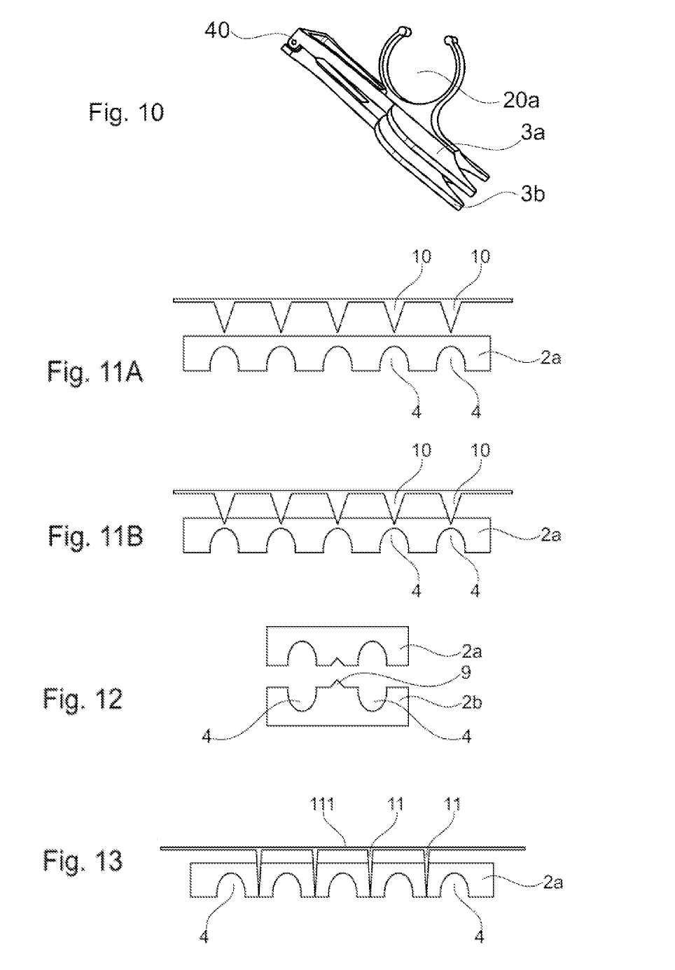

The device (1) in the form of a clamp may comprise a single housing (20a) for receiving a finger, as illustrated in FIG. 10, or two housings, as previously described.

It may be possible for the housing(s) (20a, 20b) to be oriented rotationally, which enables an adjustment according to the angle between the user's thumb and index finger. They may be positionable, where appropriate, at various points (60a), (60b) or (60c), depending on the desired distance between the fingers and the face of the user, as illustrated in FIG. 8.

The mould (2a, 2b) may be integrated into the jaws (3a, 3b). The device (1) in the form of a clamp may then be made of a flexible material of silicone type for example, in order to facilitate demoulding.

The mould may also be made of a flexible material of silicone type with the inclusion of a rigid frame. The housing(s) (20a, 20b) may be made of a rigid material, in order to improve the holding of the clamps in place and the overall shape of the mould, while facilitating demoulding.

It is possible for the mould (2a, 2b) not to be integrated into the jaws (3a, 3b), the jaws carrying the mould, which is then added to the jaws. The mould may be flexible and held on the jaws for example by adherence or by matching shapes, for example by click-fastening and/or sliding attachment. The jaws are then preferably rigid.

The mould (2a, 2b) may or may not be disposable. The jaws (3a, 3b) may or may not be reusable.

The composition (6) may be present within the mould before use of the device (1), in the part (2a), the part (2b) or in both parts. The loading of the mould with composition may be carried out prior to the use of the device, for example using a metering device.

The device in the form of a clamp has the advantage of being light, of allowing the user to precisely perceive the stresses applied, to the eyelid, during the operations, of creating a very small visual eclipse allowing optimal vision for the user, and of being easy to use.

The device (1), whether or not it is in the form of a clamp, may comprise a system which facilitates demoulding, for instance a set of small blades (10) which deform the mould, for example when moved relative to said mould in the direction of the imprints (4), as illustrated in FIGS. 1A and 11B.

The mould (2) may also comprise, as illustrated in FIG. 12, reliefs (9) between two imprints (4) intended to form two cavities, so as to cut any bridges of composition.

The device (1) may comprise blades (11) which serve to cut any bridges of composition between two cavities, as illustrated in FIG. 13.

The blades (11) are, for example, retracted within the part of the mould (2a) before closing thereof, and tend to advance upon closing of the mould. The blades (11) are, for example, interlinked with a support (111) which is mobile relative to the part (2a).

As a variant, the material of the part (2a) is elastomeric and overmoulded on the blades (11), which can push on the bridges of composition extending between the imprints (4) by virtue of the deformability of the material with which the part (2a) is made.

Other systems can be further used for cutting the bridges, for example a system which makes it possible to send compressed air via slits located between the imprints or a heating element.

The composition (6) can make it possible to attach an additional fibre (12) to an eyelash (7), with or without axial overlap between the eyelash and the additional fibre, as illustrated respectively in FIGS. 14A and 14B. The eyelash may be on the additional fibre if the additional fibre is in a cavity of the lower mould part, or vice versa, if the additional fibres are in cavities of the upper mould part. In practice, the additional fibres may also be next to the eyelashes depending on the organization caused by the pressure of the two parts of the mould.

The additional fibres may in particular be false eyelashes.

The additional fibres (12) may be placed on hold in the imprints (4) of the mould (2) (FIG. 15A). The composition (6) may then be applied to the ends of the additional fibres (12) and the eyelashes (7) introduced into the imprints (4) of the mould (2) and brought into contact with the composition (6) (FIG. 15B).

The cavities (5) may, according to various variants, have a shape which is approximately cylindrical (FIGS. 16A and 6B), approximately spherical (FIG. 16D), cylindrical on a part of their length (L) and spherical on the other part of their length (FIG. 16C), or have a decreasing cross section so as to give the eyelash(es), once moulded by the composition, a tapered appearance (FIG. 16B).

They may also have a semi-cylindrical shape in the case where the part (2a) of the mould is flat and the part (2b) of the mould is semi-cylindrical in the cavity (5) formation zone (FIG. 6A).

The depth (j) of the imprints 4 may range between 75 μm and 1.5 mm.

Each cavity (5) can receive one or more eyelashes (7), as illustrated respectively in FIGS. 17A and 17B.

Before moulding, the composition (6) may, according to various variants, be present on only one part of the cavity (5), as represented in FIGS. 18A to 18C, on all of the cavity, as illustrated in FIG. 18E, or absent from the cavity as represented in FIG. 18D. In the latter case, the composition may be injected via an injection channel (8) communicating with the cavities (5) of the mould, as illustrated in FIG. 19.

The composition (6) may be present initially, before putting the eyelashes in place, on the upper part of the cavity, corresponding to the part (2a) of the mould (FIG. 18A), on the lower part of the cavity, corresponding to the part (2b) of the mould (FIG. 18B), or both on the upper part and on the lower part of the cavity, corresponding to both parts (2a) and (2b) of the mould (FIGS. 18C and 18E).

The device (1) may comprise, or be placed in proximity to, a heating element (30), as illustrated very diagrammatically respectively in FIGS. 20A and 20B, in order to aggregate the particles in the dispersed state and/or to evaporate the liquid medium, the heating element (30) being, for example, a resistive element, a radiative element or a wave dispenser. The device may provide for the injection of the composition, in particular by an injection channel (8), as represented in FIG. 19.

The device (1) according to the invention may comprise a triggering system, such as a light element (32), as represented respectively in FIGS. 22A and 22B, for example an IR, UV or visible light illuminator. The mould (2) is then preferably transparent.

The device may comprise, or be placed in proximity to, a system (31) which assists drying by providing energy, in particular in the form of light or heat, and/or which comprises an aeration circuit, suction and/or blowing of air, as illustrated respectively in FIGS. 21A and 21B, in order to evaporate at least a part of the liquid medium.

The mould (2) may comprise an absorber specific for the liquid medium in order to eliminate at least a part of said liquid medium.

### EXAMPLES

### Example 1: Assembly Containing a Composition Based on Wax and Latex and a Device which has a Heating Element

1) Solidifiable Cosmetic Composition

A composition in accordance with the invention is prepared using the weight proportions described below.

The composition is fluid at ambient temperature.

| | % by weight (relative |
|---|---|
| | to the total weight of |
| Compounds | the composition) |
| Aqueous microdispersion of carnauba wax | 50 |
| sold under the name Mexoryl SAP by the company | |
| Chimex | |
| Latex Styrene/Acrylate/Ammonium Methacrylate | 50 |
| copolymer sold under the name Syntran 5760 | |
| CG by the company Interpolymer | |

2) Device

The device used in Example 1 is as described in FIGS. 1A and 1B.

It contains two mobile jaws (3a) and (3b), one of the two jaws having a temperature-controlled heating system.

The two parts (2a) and (2b) of the mould, placed between the two jaws, are made of cross-linked silicone elastomer and are 2 mm thick. They each comprise 20 grooves approximately 0.5 mm in diameter over a length of 2.5 cm.

The two jaws comprise two lugs and the two parts of the mould each comprise two housings which fit the lugs.

When the two parts of the mould are brought together, 20 leaktight cavities, closed at their end, are created on the sides.

3) Cosmetic Assembly

The solidifiable cosmetic composition based on wax and latex is placed in the cavities and grooves of the two parts (2a) and (2b) of the mould.

The two jaws (3a) and (3b) containing the two parts (2a) and (2b) of the mould are moved near to the eyelash fringe and then closed so as to trap it.

The heating is activated by virtue of the heating element of the device (1) for 5 minutes until a temperature of approximately 70° C. is reached.

The heating is then stopped and the assembly is left to cool for 10 minutes. The two jaws (3a) and (3b) are then opened. The cosmetic composition has become solid by aggregation of the particles during the increase in temperature.

The two parts (2a) and (2b) of the mould remain attached to the eyelash fringe. The two parts of the mould are therefore moved apart, by pulling along their width, in order to release the eyelashes.

The result is then slightly rubbed in order to break the possible bridges of composition formed between two imprints.

A homogeneous makeup result is obtained on the eyelashes. The eyelashes are coated with a smooth, homogeneous and cohesive deposit.

### Example 2: Assembly Containing a Composition Based on Latex and Ethanol and a Device which has a Heating Element

1) Solidifiable Cosmetic Composition

A composition in accordance with the invention is prepared using the weight proportions described below.

The composition is fluid at ambient temperature.

| | % by weight (relative |
|---|---|
| | to the total weight of |
| Compounds | the composition) |
| Latex Polyester-5 sold under the name Eastman | 40 |
| AQ 55 S Polymer by the company Eastman | |
| Chemicals | |
| Ethanol | 20 |
| Water | qs 100 |

2) Device

The device used is identical to that of Example 1.

3) Cosmetic Assembly

The solidifiable cosmetic composition based on latex and ethanol is placed in the cavities and grooves of the two parts (2a) and (2b) of the mould.

The two jaws (3a) and (3b) containing the two parts (2a) and (2b) of the mould are moved near to the eyelash fringe and then closed so as to trap it.

The heating is activated by virtue of the heating element of the device (1) for 5 minutes until a temperature of approximately 70° C. is reached.

The heating is then stopped and the assembly is left to cool for 10 minutes. The two jaws (3a) and (3b) are then opened. The cosmetic composition has become solid by aggregation of the latex particles during the increase in temperature.

The two parts (2a) and (2b) of the mould remain attached to the eyelash fringe. The two parts of the mould are therefore moved apart, by pulling along their width, in order to release the eyelashes.

The result is then slightly rubbed in order to break the possible bridges of composition formed between two imprints.

A homogeneous makeup result is obtained on the eyelashes. The eyelashes are coated with a smooth, homogeneous and cohesive deposit.

1.-28. (canceled)

29. Cosmetic assembly, intended for the treatment of keratin fibres, comprising:
a fluid cosmetic composition containing at least particles in the dispersed state in a liquid medium, said composition being solidifiable via the aggregation of said particles; and
a device comprising a mould, the mould comprising at least one cavity, into which or each of which at least one of said fibres can be at least partially introduced for the purposes of moulding said composition onto at least one part of said fibre(s) present in the cavity or cavities.

30. Assembly according to claim 29, wherein the aggregation of the particles dispersed in the liquid medium is induced by at least partial, or even total, evaporation of said liquid medium.

31. Assembly according to claim 29, wherein the evaporation of said liquid medium is carried out at a temperature of between 45° C. and 90° C.

32. Assembly according to claim 29, wherein the cosmetic composition comprises from 10% to 55% by weight of particles in the dispersed state, relative to the total weight of the composition.

33. Assembly according to claim 29, wherein the cosmetic composition has a viscosity of less than 100 Pa·s, at ambient temperature and pressure.

34. Assembly according to claim 29, wherein the particles in the dispersed state are chosen from a wax, a film-forming polymer, and mixtures thereof.

35. Assembly according to claim 29, wherein said liquid medium is chosen from water, ethanol or mixtures thereof.

36. Assembly according to claim 29, wherein the mould comprises at least two cavities.

37. Assembly according to claim 29, wherein the cavity or cavities is (are) formed by bringing two jaws together.

38. Assembly according to claim 29, wherein the device comprises at least one heating element which serves to increase the temperature of the cosmetic composition, the increase in the temperature inducing solidification of the composition.

39. Assembly according to claim 29, wherein the device comprises at least one system which assists the drying of the cosmetic composition, the system providing energy, and/or comprising an aeration circuit, suction and/or blowing of air.

40. Assembly according to claim 29, wherein the device comprises at least one system for admitting material, which serves to introduce a part or all of the cosmetic composition into the mould cavity or cavities.

41. Assembly according to claim 29, wherein the mould is disposable.

42. Assembly according to claim 29, wherein the device comprises one or more additional fibres, the cosmetic composition ensuring the attachment of the additional fibre(s) to the keratin fibre(s), with or without axial overlap between the two types of fibres.

43. Assembly according to claim 42, wherein the additional fibre(s) are prepositioned in the mould cavity or cavities or interlinked with a support which makes possible to preposition them.

44. Assembly according to claim 29, each cavity constituting a space which is substantially entirely closed, in the absence of keratin fibre.

45. Assembly according to claim 29, each cavity delimiting a space which is closed with the exception of one end via which the keratin fibre(s) which are at least partially introduced into the cavity communicate with the exterior.

46. Process for cosmetic treatment of one or more keratin fibres, comprising at least the step consisting in moulding a fluid cosmetic composition, containing at least particles in the dispersed state in a liquid medium, said composition being solidifiable via the aggregation of said particles, onto at least one part of said fibres, by means of one or more cavities, of a mould, into which said fibres are at least partially introduced.

47. Process according to claim 46, wherein said fibres are, optionally individually, at least partially introduced into said cavities.

48. Process according to claim 46, wherein the entire composition or part of the cosmetic composition is deposited on just one part of the length of at least one of said fibres.

49. Process according to claim 46, wherein the cosmetic composition is introduced in the liquid state into the mould so as to subsequently solidify therein.

50. Process according to claim 49, wherein the cosmetic composition is introduced in the liquid state into the mould so as to subsequently solidify therein, via the aggregation of the particles dispersed in the liquid medium, the aggregation being induced by partial or total evaporation of said liquid medium.

51. Process according to claim 46, wherein the cavity or cavities of the mould are formed by bringing two jaws together.

52. Process according to claim 46, wherein the cosmetic composition is moulded onto at least one part of at least one of said keratin fibres and of at least one additional fibre, the cosmetic composition ensuring the attachment of the additional fibre(s) to the keratin fibre(s), with or without axial overlap between the two types of fibres.

53. A molding process of a material in at least one cavity of a mould, for the purposes of depositing on keratin fibres, comprising at least a step of application of said mould to said fibres, wherein the material consists in a fluid cosmetic composition containing at least particles in the dispersed state in a liquid medium, said composition being solidifiable via the aggregation of said particles.
